(19) United States
(12) Patent Application Publication
(10) Pub. No.: US 2025/0235341 A1
(43) Pub. Date: Jul. 24, 2025

(54) ORTHOSIS WITH ADJUSTABLE LOAD BEARING

(71) Applicant: Technion Research & Development Foundation Limited, Haifa (IL)

(72) Inventors: Dana SOLAV, Haifa (IL); Etamar BAREKET, Haifa (IL); Hadas HUBER, Haifa (IL); Lee Jordan DOTAN, Haifa (IL); Eshraq SAFFURI, Haifa (IL)

(73) Assignee: Technion Research & Development Foundation Limited, Haifa (IL)

(21) Appl. No.: 18/757,131

(22) Filed: Jun. 27, 2024

Related U.S. Application Data
(60) Provisional application No. 63/623,434, filed on Jan. 22, 2024.

(51) Int. Cl. A61F 5/01

(52) U.S. Cl. CPC A61F 5/0111

(57) ABSTRACT

An orthosis apparatus for offloading weight from a foot of a patient, comprising: a leg brace, sized and shaped to attach to an area above the ankle and at least one foot support component, comprising a contact plate, wherein the at least one foot support component is connected to the leg brace; wherein the contact plate comprises a convex curve shaped region, sized and shaped to provide a rolling motion when contacting a surface; wherein the contact plate is positioned at the side of the foot, at a height which can be selected to be above, below or at the bottom of the foot surface; and wherein the orthosis apparatus is configured to offload weight by providing support extending downwards to the contact plate.

### RELATED APPLICATION

This application claims the benefit of priority under 35 USC § 119 (e) of U.S. Provisional Patent Application No. 63/623,434 filed on Jan. 22, 2024, the contents of which are incorporated herein by reference in their entirety.

### FIELD AND BACKGROUND OF THE INVENTION

The present invention, in some embodiments thereof, relates to an orthosis device configured to provide support whilst optionally keeping a close to normal walking gait, and, more particularly, but not exclusively, to an ankle-foot orthosis configured to provide adjustable load bearing between the foot and the leg area above the ankle, whilst optionally keeping a close to normal walking gait, in accordance with the patient's changing needs.

Various medical conditions, such as diabetic foot ulcers and ankle fractures, may prevent individuals from loading their ankle and/or foot. To date, crutches are commonly used to ambulate with unilateral ankle and foot offloading. Crutches allow greater mobility and functionality with respect to wheelchairs. However, they severely alter the natural walking pattern, overstrain the upper body, and might cause pain and secondary injuries. Alternative devices have been proposed that allow ankle-foot offloading while freeing the upper extremities. Among these, are knee crutches, e.g., iWalk [Hackney et. al., Foot & Ankle Orthopaedics, 2022, Vol. 7 (4) 1-8] and other ankle-foot orthoses designed to reduce weight on the ankle/foot by transferring it to the patient's leg and calf area. See for example the review of commonly used types and recent development of ankle-foot orthosis (AFO) [Choo, Y. J.; Chang, M. C., Healthcare 2021, 9, 1046] and other AFO's such as described in U.S. Pat. Nos. USD635,269S1; 8,403,872B2; U.S. Patent Application Publication No. US20150080777; U.S. Patent Application Publication No. US20100106065A1; International Patent Application No. WO2023278961; U.S. Pat. No. 7,666,155B1; U.S. Patent Application Publication No. US20170056230A1 and International Patent Application No. WO2022020769.

### SUMMARY OF THE INVENTION

According to an aspect of some embodiments of the present invention, there is provided an orthosis apparatus for offloading weight from a foot of a patient, comprising: a leg brace, sized and shaped to attach to an area above the ankle; and at least one foot support component, comprising a contact plate, wherein the at least one foot support component is connected to the leg brace; and wherein the contact plate comprises a convex curve shaped region, sized and shaped to provide a rolling motion when contacting a surface.

In some embodiments, the contact plate is positioned at the side of the foot, at a height which can be selected to be above, below or at the bottom of the foot surface; and
wherein the orthosis apparatus is configured to offload weight by providing support extending downwards to the contact plate.

According to an aspect of some embodiments of the present invention, there is provided an orthosis apparatus for offloading weight from a foot of a patient, comprising a leg brace, sized and shaped to attach to an area above the ankle; at least one foot support component, comprising a contact plate configured to contact a walking surface; and a strut, upright connecting the leg brace to the foot support component at a certain length between the brace and the foot support component, wherein the contact plate is positioned at the side of the foot; and wherein the orthosis apparatus is configured to offload weight by providing support extending downwards to the contact plate.

In some embodiments, the certain length between the brace and the foot support component is adjustable so as to position the contact plate at a height which can be selected to be above, below or at the foot plantar surface.

In some embodiments, the contact plate comprises a convex curve shaped region, sized and shaped to provide a rolling motion when contacting a surface.

In some embodiments, the convex curved shaped region is shaped and sized to provide a rolling motion in the sagittal plane that reproduces the roll-over shape (ROS) obtained by a non-injured leg during walking.

In some embodiments, the curve shaped region comprises at least two distinct curved regions having a different degree of curvature deviation from an axis.

In some embodiments, the contact plate additionally comprises a frontal plane oriented-curve configured to reproduce a medial-lateral shift in a center of pressure during walking.

In some embodiments, the brace is fabricated to fit a patient's leg by use of computer generated design based on scanning the leg.

In some embodiments, the orthosis apparatus comprises at least 2 foot support components.

In some embodiments, the area above the ankle is located on the thigh.

In some embodiments, the area above the ankle is located on the shank.

In some embodiments, the overall curvature of the curve shape is between 0.1 and 0.8 times the patients' height.

In some embodiments, the device further comprises a means to measure and/or monitor the amount of load offloaded from the foot to the leg.

According to an aspect of some embodiments of the present invention, there is provided a method of offloading weight from a foot of a patient, the method comprising: suspending the leg of the patient at an area above the ankle in an orthosis apparatus brace, wherein the foot is positioned below the knee; providing support at the side of the foot with a foot support component having a contact plate, situated at the side of the foot and connected by a strut to the brace, wherein the support is provided downright from the brace to the contact plate, thereby offloading weight from the foot to the area above the ankle.

In some embodiments, the method further includes supporting at least two sides of the foot.

In some embodiments, the method further includes adjusting the offloading of weight between the foot and the area above the ankle by positioning the contact plate at a height relative to 1-2 millimeter steps over or under the height of the foot plantar surface.

In some embodiments, the method further includes adjusting the offloading of weight between the foot and the area above the ankle by positioning the contact plate at a height relative to about the height of the foot plantar surface, wherein said height effects an offload of between 200 gr and 2 kg off the foot over at least 10% of movement.

In some embodiments, the area above the ankle is located on the thigh.

In some embodiments, the area above the ankle is located on the shank.

In some embodiments, the method further includes measuring and/or monitoring the amount of load transmitted to the foot and/or to the shank.

In some embodiments, the contact plate comprises a convex curve shaped region, sized and shaped to provide a rolling motion when contacting a surface.

In some embodiments, the method further includes adjusting the offload of weight by:
Positioning the contact plate at a height below the foot plantar surface thereby offloading all of the weight to the area above the ankle;
Positioning the contact plate at a height above the foot plantar surface thereby offloading all of the weight to the foot; or positioning the contact plate at a selected height at about the level of the foot plantar surface, thereby offloading part of the weight from the foot to the area above the ankle; wherein adjusting the height of the contact plate is effected by changing the length between the brace and the foot support component.

In some embodiments, the selected height at about the level of the foot plantar surface, ranges between 30 millimeters over to 30 millimeters under the plantar foot surface.

In some embodiments, the contact plate comprises a convex curve shaped region, sized and shaped to provide a rolling motion when contacting a surface.

In some embodiments, the adjusting is provided in steps in accordance to a disease progress, exercise needs and/or rehabilitation level.

In some embodiments, the adjusting of offload step is provided for a defined time frame.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as custom made preparation of device to fit an individual, assessing individual walking gait pattern, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying images or drawings. With specific reference now to the images or drawings in detail, it is stressed that the particulars shown are by way of example and for purposes of illustrative discussion of embodiments of the invention. In this regard, the description taken with the images or drawings makes apparent to those skilled in the art how embodiments of the invention may be practiced.

In the drawings:

FIGS. 1A-D present an exemplary AFO (ankle-foot orthosis) according to some embodiments of the present invention;

FIGS. 2A-E present an exemplary AFO and exemplary use of an AFO according to some embodiments of the present invention;

FIGS. 3A-F present an exemplary process of design of an AFO component according to some embodiments of the present invention;

FIGS. 4A-D present an exemplary AFO component according to some embodiments of the present invention;

FIG. 5 presents an illustration of an exemplary component fabrication process according to some embodiments of the present invention; and

FIGS. 6A-J present an exemplary process of design and fabrication of an AFO according to some embodiments of the present invention.

### DESCRIPTION OF SPECIFIC EMBODIMENTS OF THE INVENTION

The present invention, in some embodiments thereof, relates to an orthosis device and, more particularly, but not exclusively, to an ankle-foot orthosis configured to provide adjustable load division between ankle and device and preserving the natural motion during load bearing on an injured limb.

An aspect of some embodiments of the invention relates to allowing a rolling motion during movement with an injured foot of a patient. In some embodiments of the invention, an orthosis device including a foot support which can contact the ground is provided and the foot support has a complex shape at the bottom thereof. In some embodiments of the invention, the foot support defines a contact plate (which may be narrow and blade-like) for contacting the ground.

In some embodiments the contact plate includes at least one curved section selected to enables a rolling motion when contacting a surface. In some embodiments of the invention, a desired roll-over shape (ROS) is selected for the patient. For example, the ROS may be selected to encourage and/or support, the (injured) lower limb's ability to roll forward during a single-limb support phase of human walking. It is a particular benefit of some embodiments of the invention that providing a shaped contact plate (e.g., using a ROS curve) can allow a natural gait pattern even in the presence of degraded or lacking of ankle flexion and/or ability to support full body weight with the foot or ankle.

In some embodiments the bottom surface of the support defines a curve in the sagittal and/or lateral plane. In some embodiments the support is configured in a convex curve shape so as to provide a rolling motion during movement.

In some embodiments, the curve is configured to reproduce the roll-over motion obtained during walking. This means that the trajectories of the position of the foot center of pressure (CoP) relative to the body during healthy walking remain the same (e.g., within a distance of 5 cm, 3 cm, 1 cm or less) when replaced with the AFO that is equipped with the ROS, for at least 30%, 50%, 90% or intermediate percentages of a foot contact trajectory. Optionally, desired CoP is determined by measuring the healthy leg and/or using biomechanical considerations. Optionally or additionally, the CoP may be found in an iterative method by modifying the curve and/or position of the curve relative to the leg until a desired gait is found. It is noted that in some cases, it may be desired to give a non-normal gait, for example, due to other abnormalities of the patient, such as joint damage or varying limb length or muscle weakness.

This potentially allows the offloaded leg to move similarly to the healthy walking pattern irrespective of the changing angle and flexibility or fixture of the ankle joint.

In some embodiments the roll over curve refers to a convex curve shape that enables a rolling motion of the device during walking movement when using the apparatus. In particular, the curve may include one or more linear sections. In some embodiments of the invention, the curve is approximated by piecewise section which may be, for example, curved, and/or straight. In some embodiments of the invention, one or more gaps are provided in the curve. In some embodiments of the invention, at least a part of the curve is provided as spikes or short sections with gaps and rolling motion is provided by weight being transferred from one spike to the next, as the orthosis is rolled. A potential advantage of providing gaps is that one or more such short segment or spike can be adjusted without modifying the entre curve. For example, one or more such spikes may be provided by screws whose head contacts the ground and screwing in or out changes their distance from the bottom of the foot support to the ground, thereby defining how the foot will roll.

In some embodiments of the invention, the contact surface is in the form of a blade on one side of the foot. This has the potential advantage that foot movement is not necessarily constrained by the contact surface (though constraint using elastic element and/or straps or netting may be desirable). Another potential benefit lies in partial foot pressure. Depending on the design of the curve (e.g., relative to the foot geometry and/or ankle function), different parts of the roll over motion may include contact of the human foot with the ground. Depending on the relative altitude (in a direction away from the ground) at that point of contact, contact can be with the orthosis, the foot, or a combination. This means that the contact of the foot with the ground can be limited to certain parts of the roll over. When the contact is a combination of foot and orthosis contact, small changes in the altitude (and/or elasticity of the orthosis) can define the percentage of the contact force borne by the foot. This may allow gradual load bearing during rehabilitation.

In some embodiments of the invention, the contact surface and the orthosis is rigid and/or rigidly attached to the limb. Alternative, some elasticity may be provided, for example, the connection of the contact surface to the rest of the orthosis may be elastic, individual parts of the contact surface may be also elastically connected, parts of the contact surface may be elastic and/or the contact surface may be covered with an elastic section. For example, a foam layer or insert may be provided on some of the contact surface and/or a spring coupling may be provided between part of the contact surface and the rest of the orthosis. Some amount of elasticity may be provided by the geometry and material properties of the material the orthosis is made of, for example, metal.

In some embodiments of the invention, the contact surface is in the form of a relative thin blade (e.g., less than 1 cm in width). Optionally, two such blade are provided, one on either side of the foot. If the curve is not identical on the two blades and/or not aligned, the orthosis roll over can be used to provide lateral movement. In other embodiments, the contact surface lies under the foot.

In some embodiments of the invention, the contact surface extends enough to support roll over of both heel movement and toe launch. Optionally, the orthosis extends enough to protect the heel and/or ankle and/or toes from bumping against objects.

In some embodiments of the invention, the orthosis includes a mechanism, such as an elastic strap, to help overcome foot drop.

In some embodiments a cover may be added to the contact plate, optionally, the covers may be used to add friction to the contact plates so as to prevent sliding of the leg during usage, optionally or additionally the covers may provide guarding against wearing the contact surface, and can be replaced once worn out. In some embodiments covers may be fabricated to provide additional fine tuning of the contact plate ROS, a potential benefit of such covers is that they may be easily replaced and fitted to the contact plate during continuing use or as required during the healing process, without the need to replace the contact plate.

In some embodiments, the ROS curve (especially for matching a correct or otherwise desired gait) is determined using computer aided design and the contact surface may be manufactured automatically form such design. Input to the design is option patient gait parameters, captured from this or other patients.

An aspect of some embodiments of the invention relates to supporting the foot medially and/or laterally rather than directly under the foot, for example using an orthosis device configured to provide support extending downwards from a leg to the side of the foot. Such support can be given when the apparatus is configured to offload weight by providing support extending downwards to the contact plate. A potential benefit of such lateral support is that the foot can be positioned as close as desired to the ground surface which may allow the injured leg to have an effective length close or identical to that of the contralateral non-injured leg. Another potential benefit is the enablement of using a foot support configured with a ROS curve without having to increase the height of the injured foot, since if a proper ROS curve were implemented under the foot, the height increase may be too large.

Natural gait, or the normal walking pattern of an individual is typically determined by parameters like pelvic rotation, pelvic tilt, knee flexion, foot and ankle motion, knee motion and lateral displacement of the hip. Gait symmetry is known to be an indicator of healthy movement and prevents further injuries to the back and upper body. In some embodiments, natural gait can be potentially maintained or approximated utilizing a roll-over shape (ROS) that is inferred from natural gait parameters and potentially provides that symmetric kinematics is achieved. Additionally, this unique support method potentially allows a natural symmetric setting of the injured leg during movement, potentially avoiding discomfort and/or pain due to a non-balanced division of load between the injured and healthy leg and potentially allowing the ground reaction force (GRF) pattern to remain similar to normal gait, despite the full, or optionally partial load reduction from the foot and ankle. This may also allow rehabilitation to focus on correct walking gait and/or avoid learning incorrect gait habits during recovery and/or rehabilitation.

Additionally, the absence of support at the bottom of the foot potentially enables a division of weight bearing between the device and the foot by optionally enabling the plantar foot bottom surface to contact the ground surface, however, only at some times, parts of gait and/or with a limited contact force. Another potential advantage is allowing various footwear or medical dressing, casts, or fixations including with different sole thicknesses (e.g., 0-3 cm) and including with different sole characteristics (e.g. plasticity, flexibility and/or softness) to be used (and replaced) with the injured foot. Also, it is noted that lateral support may allow more or most of the foot to be exposed, e.g., to air flow and/or treatment and/or to allow putting on and removing footwear. It is noted that the orthosis according to some embodiments of the invention can accommodate the patient's existing or preferred footwear.

An aspect of some embodiments of the invention relates to an adjustable load bearing between an injured foot and an orthosis device mounted thereon. In some embodiments of the invention, this adjustment is by positioning a foot support bottom surface of the device at a height selected to be above, at or below the plantar foot surface level, optionally taking into account ankle flexion during walking (or other gait type as may be desired).

In some embodiments, the orthosis device can be adjusted to optionally provide change of the load bearing on an injured foot, for example, gradual changes over time in such load bearing in accordance with the patient's ongoing progress and/or acute change in accordance to need (such as an inflammation, pain or weakness or physiotherapy exercises). In rehabilitation processes, gradual return to foot and ankle loading can be particularly advantageous in terms of recovery time and maintaining muscle and bone health during the offloading period. Adjustment can be performed in according to healing protocol, e.g., gradual changes in one direction or different adjustments for different stages of the recovery process or different time frames during a day.

In some embodiments the height of the foot support bottom surfaces can be adjusted independently at each side of the foot to provide a change in pronation/supination, which may affect leg angle and/or encourage better foot placement.

An aspect of some embodiments of the invention relates to designing an orthosis device comprising a leg brace, optionally including alignment and connectors to struts. In some embodiments of the invention, the leg brace may be designed using a 3D scan file of the leg. A potential benefit of exact fitting based on a scan is reduction or avoidance of discomfort and/or pain due to inaccurate fitting of the brace to the leg, e.g. the shank and/or fast attachment of the brace to the leg.

In some embodiments, the leg brace can be sized and shaped to attach to a desired area above the ankle, e.g. the shank or the thigh. Optionally, the attachment can be to more than one area of the leg (e.g., at more than one spaced apart axial location along the leg).

An aspect of some embodiments of the invention relates to assembling an orthosis device from a plurality of parts, wherein some of the parts are ready made and some are customized, for example, some are customized to specific subsets of patients, in accordance with pre-determined properties and/or some are personally customized to the specific patient.

In some embodiments specific parts can be changed during the course of patient healing process or changing needs, e.g., changes in attachment area or surface, changes in contact curve and/or to accommodate changes in ground surfaces (e.g., carpet, wood, stone/ceramic flooring or outdoor surfaces.

In some embodiments additional components can be added to the orthosis device for different purposes, e.g. sensors for collecting and monitoring data (e.g., ROS, ground reaction force (GRF) or optionally walking gait parameter related data or disease related data). Optional means to monitor amount of load or offload from foot to leg, means to monitor foot and thigh angles for accurate adjustment, temperature, humidity pulse sensors, blood sugar sensors, or other sensors for chemicals released by sweat).

The principles and operation of some embodiments of the present invention may be better understood with reference to the figures and accompanying descriptions.

Before explaining at least one embodiment of the invention in detail, it is to be understood that the invention is not necessarily limited in its application to the details set forth in the following description or exemplary implementation. The invention is capable of other embodiments or of being practiced or carried out in various ways.

### Components

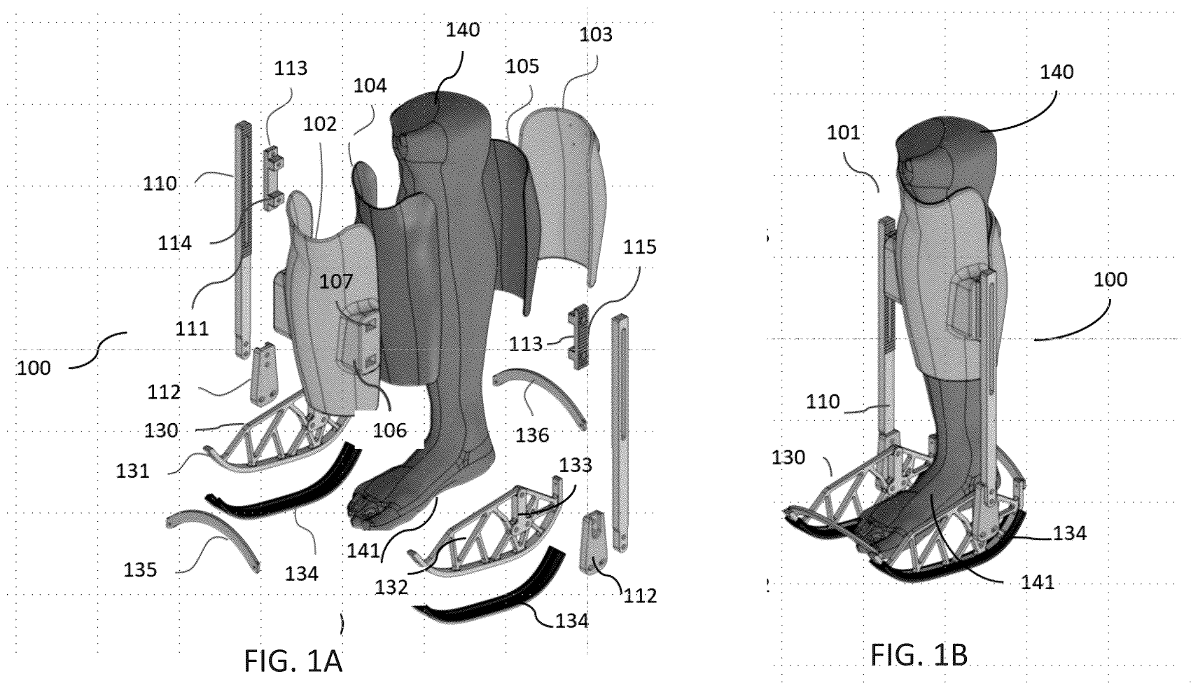

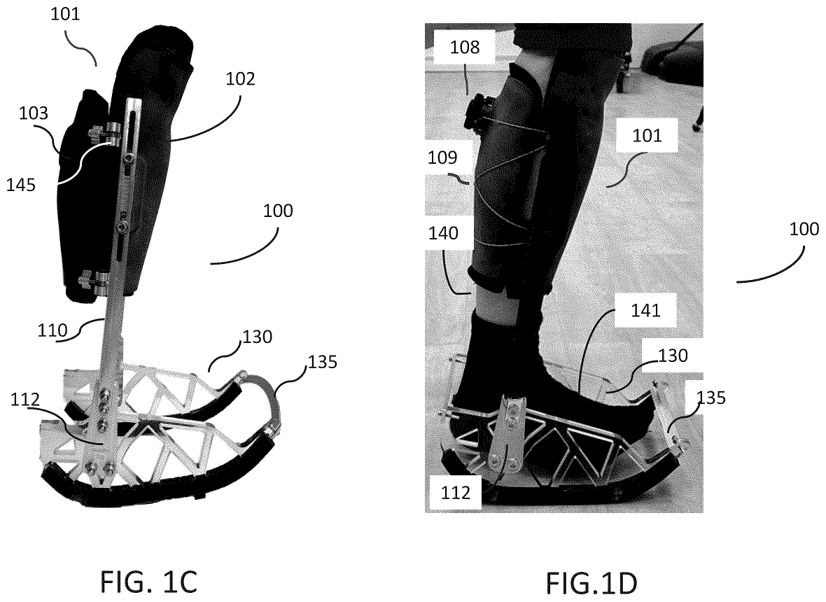

Referring now to FIGS. 1A-D, which show an exploded view of an exemplary orthosis 100 (FIG. 1A), a computer graphics showing of orthosis 100 mounted on a leg 140 (FIG. 1B), a side image of orthosis 100 (FIG. 1C) and a side image of orthosis 100 partially covered and mounted on a live foot 141 (wearing a sock) (FIG. 1D), in accordance with some embodiments of the invention.

An exemplary orthosis device 100 includes a brace 101, which in a particular embodiment can be a shank brace 101 and a strut 110 or other component interconnecting the part of device 100 that is attached to the leg to a foot support plate 130. FIG. 1A-1D show a more detailed design, but it should be noted that this is only one possible embodiments, and as will be described, it is sufficient to have only these three components. If there is a different mechanism attached to the leg, for example, an Ilizalarov frame, then the brace and/or the shank may be omitted, reusing what is already mounted on the patient

The shank brace 101 can be comprised of anterior 102 and posterior 103 parts, each can be designed separately so to provide a secure fit to the shank whilst enabling knee movement. Optionally, the shank brace can be made of a single ring-like unit worn on the leg. The shank brace is optionally fabricated from a relatively strong material so as to provide support to and guard the leg. Exemplary materials that can be used for the shank can be polyamide, e.g., PA12, carbon fiber, or fiberglass.

An anterior 104 and a posterior 105 soft liner can provide the option to secure the fit of the brace 101 without harming or irritating the skin, and help distribute the loads (particularly shear stresses and pressures) across the skin and soft tissue. The soft liners can also optionally be easily cleaned and/or replaced as needed. In some embodiments, liners made with different thicknesses, mechanical properties, or adhesive properties can be used, e.g., neoprene, silicone, polyurethane, or other elastomers.

Other materials can be selected, added or replaced to better fit the brace 101, for example in case the volume and/or shape of the body part has changed (as it may change due to injury, muscle wasting and/or rehabilitation), or in the case that a lining is needed to guard the skin from friction by the brace 101. In specific embodiments the brace 101 can be custom made to be attached to a desired area on the leg 140, above the ankle, to provide the best fit to a specific patient, limb area and optimal location on the skin surface. An area above the leg can be for example the upper or lower part of the shank or calf, or the upper or lower part of the thigh. In specific embodiments, more than one brace can be used, for example at spaced apart axial locations. This may allow to attach the orthosis device to the leg for various needs, e.g. knee health and/or skin health.

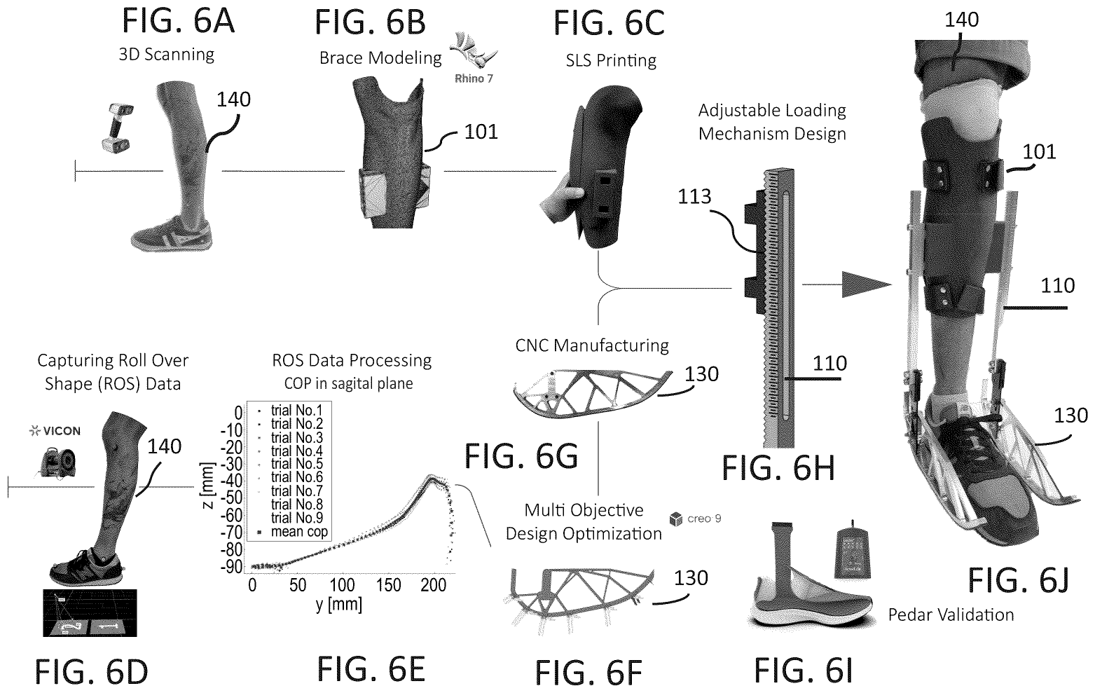

A tightener 108, for example, as used in footwear, can be used to align and fasten the posterior and anterior parts of the shank brace 101 and soft lining 102, 103, 104, 105 to each other and to the leg 140. For example, a ribbon, strap, cord or thread, can be used to wrap around the brace, as shown for example a ribbon 109 in FIG. 1D, a buckle 145 in FIG. 1C and straps in FIG. 6J. The ribbon 109 can then be adjusted and stretched to further secure the binding and adjust the fitting tightness as desired. Other means of adjusting the brace fitting pressure can include for example, hook-and-loop straps, belts, buckles, ratchets, ladder or straps.

A strut 110 can be used as a connecting structure. The strut can be elongated and can connect the shank brace 101 to the foot support 130. Optionally, the connection between strut 110 and brace 101 can be provided by at least one bracket 113. Optionally, the bracket 113 is fitted to brace 101 by attaching to a suitable brace connector 106 that may have fitting structure 107 to connect the bracket 113. Optionally, bracket 113 has a shape which matches the shape of the brace 101 or structure 107 and/or includes one or more protrusions 114 or recesses which have a matching geometry on the brace 101 and/or structure 107, e.g., for interlocking and providing load bearing. Optionally or additionally, one or more screw holes or inserts are provided on structure 107 and/or brace 101 for attached bracket 113. In some embodiments of the invention, bracket 113 is formed as an insert in brace 101 or structure 107 (e.g., during molding). In some embodiments, strut 110 is attached directly to brace 101.

In some embodiments of the invention, the distance between the shank brace 101 and the foot support 130 may be adjusted by changing the length of the connecting part of the strut. In one option the change in length can be achieved by changing the point along the strut which connects to the bracket 113. Optionally, as is shown for example in FIG. 1A-C, the strut 110 and the bracket 113 comprise serrated regions, 111 and 115 which are configured to fasten to each other in different locations along the strut's serrated region 111 and 115. In other embodiments, other means of changing the distance between shank brace 101 and foot support can be used, for example, strut 101 may be telescopic. In some embodiments of the invention, strut 101 is elastic, optionally only in an axial direction. Optionally, a range of motion allowed by the elasticity is limited, optionally settable, for example, using a stopping screw. The bracket 113 can optionally further be configured to provide alignment of the strut 101 to the brace 101 at different angles or positions, for example, in accordance with the patient's posture and leg positioning needs.

The strut 110 is optionally fastened to the foot support 130 by a foot support bracket 112. The foot support bracket 112 can also be configured to be used as an alignment adapter, to provide alignment of the strut to the brace at different angles or positions, for example, in accordance the patient's posture and leg positioning needs. Bracket 113 and/or 112 may be selected and/or manufactured (e.g., by 3D printing) according to a desired such alignment.

The foot support 130 may be positioned at the side of the foot, and through contacting the ground surface, the foot support can provide the offloading of weight from the foot 141 to the part of the leg onto which the brace is attached, in this example, via shank 101 to the upper part of the lower leg (e.g., the calf). A contact plate 131 at the bottom of the foot support is configured to contact the ground surface. The contact plate 131 may be curved and can optionally be configured in a roll over shape (ROS) that enables a rolling motion when contacting a surface. Optionally the contact plate 130 can be covered with a cover tread 134. Cover tread 134 may provide various mechanical properties to orthosis 100 such as added grip, cushioning, elastic give and/or prevention of damaging of floor surfaces or damaging and wear of contact plate 130. In some embodiments cover tread 134 may provide additional fine tuning of the contact plate ROS, a potential benefit of such covers is that they may be easily replaced and fitted to the contact plate during continuing use or as required during the healing process, without the need to replace the contact plate. As shown, orthosis 100 includes two contact plates 130, one on either side of the foot. Frame stiffness may be increased by providing one or more lateral connections between the parts of orthosis 100 that are on opposite sides of the leg, for example, anterior 135 and posterior 136 bridges may be added for more frame stiffness. The bridges may also, or alternatively, be used to provide a physical barrier (e.g., protection) between the injured foot 141 and the surrounding environment during movement and/or for mounting straps, netting or other structures e.g., sensors as described above for controlling and/or monitoring foot movement relative to the orthosis. While not shown, in some embodiments of the invention contact plate 131 may extend laterally (sideways) to under the foot. In one example, an arch support may extend from plate 130 to support the arch of the foot. In another example, the plate contact surface 131 extends under the foot. In another example, a strap for maintaining the foot location extends between the two plates 130. Such strap may be useful in case the patient suffers from foot drop, so as to potentially avoid undesired contact of the foot with the ground.

FIG. 1B is an illustration of the exemplary AFO components presented in FIG. 1A in an assembled configuration, showing an exemplary positioning of an injured leg within the AFO. FIG. 1C is an image of an exemplary AFO in an assembled configuration. FIG. 1D is an image of an exemplary assembled AFO manufactured prototype designed and fabricated for one individual, worn by that individual showing an exemplary positioning of an actual leg within the AFO.

In the option where the plate contact surface 131 is curved, the curve forming the ROS may comprise at least two distinct regions with different curvatures. Optionally the curve ROS comprises at least three distinct regions, where at least two of the regions have a distinct curvature. For example, a rearfoot region can be defined from the foot heel to the center of pressure during standing, and a forefoot region can be defined from the center of pressure during standing to the tip of the toes. This way, a short flat region can be introduced between the rearfoot and forefoot regions, to facilitate standing balance. Optionally, the plate contact surface 131 may comprise four, five, or more distinct regions with different curvatures, corresponding to the different phases of the walking gait cycle, namely, for example, initial contact, weight acceptance, loading response, midstance, terminal stance, and pre-swing.

Optionally, the plate contact surface 131 may comprise a three-dimensional curve (e.g., with a lateral component) representing and allowing for a medial-lateral shift in the center of pressure during walking. In some embodiments of the invention, such shift is optionally provided by differences between the curves of the two contact plates 130 or by differences in the height of each contact plate 130 as can be adjusted as desired by changing the length of the strut 110.

FIG. 1D is a picture of the manufactured prototype designed and fabricated for one individual, worn by that individual person.

By providing support at the area surrounding the foot (i.e. supporting the foot medially and laterally instead of directly under the foot), the foot 141 can optionally be located much closer to the ground, which means that the contralateral foot does not need to be substantially elevated to equate the height of both sides. This provides potential benefit for the option of having a ROS curve, since a ROS curve implemented under the foot, necessitates a height increase that would be too large and/or would not fit with existing patient footwear. Additionally, this unique support method can allow the ground reaction force (GRF) pattern to remain similar to normal gait, despite the load reduction on the foot itself. The example shown in FIGS. 1A-D shows that optionally providing one or two supports on the medial and/or lateral sides of the foot allows the foot to be much closer to the ground while still allowing a curved contact surface 131 for maintaining the roll-over motion that promotes a natural walking pattern. In specific embodiments, means for aligning between two supports can be added, so that the distance between the foot and shank on both sides of the foot is identical. In other specific embodiments the distances between foot 141 and/or foot plate 130 and shank support 101 can be adjusted to be different between the two supports.

As discussed, one potential benefit of lateral support lies in ways of adjustment of load bearing. An adjustable load mechanism that potentially enables precise control over the loading ratio between the foot 141 and the device 100 may be provided. A means for alignment can optionally or additionally be provided to keep or change the difference between the two supports 130 on each side of the leg when optionally changing or adjusting the load bearing between foot 141 and the device 100. Relative changes between the two distances can be provided for adjusting, for example, the angle of ankle and foot positioning, the relative load bearing of different parts of the foot 141, e.g., toes vs heel, or inner part vs outer part of the foot. If desired a patch may be attached to a part of the foot to make that part thicker and closer to the ground, rather to other parts. This may allow to reduce load bearing by other parts of the foot 141, while using orthosis 100.

In situations in which full and/or partial offloading of the foot 141 and ankle is required, and further in specific situations where gradual adjustment of partial offloading is required in the rehabilitation process, it can be beneficial to have an adjustable load mechanism, that can potentially enable a gradual rehabilitation.

The division of load between foot and device can optionally be adjusted as follows: as shown in FIGS. 1A-D, when the foot support plate 131 positioned under (e.g., lower than) the plantar foot surface, the support extends downwards to the side of the foot and the weight is loaded fully on the device 100. When the plate contact surface 131 of the foot support plate 130 is positioned above the plantar foot surface, the weight is loaded fully on the foot 141. When plate contact surface 131 of the foot support plate 130 is positioned at a height which is at the plantar foot surface, the support extends downwards to the side of the foot and the weight load is divided between the foot 141 and the device 100. A precise adjustment of the division of load can be potentially achieved by adjusting the height of the support plate 130 position with respect to the plantar foot surface level. As the support plate 130 is gradually positioned higher than the plantar foot surface, the weight is gradually transferred to be borne by the sole of the foot 141 to the extent that the foot sole and any optional dressing or footwear under the sole is compressed under the weight until weight is carried by the non-compressing foot surface and/or other parts of the foot 141 and leg 140, all the while keeping the injured foot braced as desired and guarded by the support plates 130 and or optionally by the support bridges (135 and 136). For example, in one protocol, a first transferred weight is capped at between 300 and 1300 grams, for example, about 1 Kg, at later times, increases in weight transfer can be, for example, in steps (not necessarily uniform) of between 300 grams and 2 kg. However, more or less weight can be transferred at each adjustment, in accordance with the patient's specific needs. Optionally, the level of transfer of weight bearing can vary along the foot depending on the design of the ROS curve. It is noted that the term “above” and “below” can change based on patient flexion of their ankle. Further, depending on walking pattern and/or such flexion and/or load preferences, a specific part of a foot may be sometimes above and sometimes below the plate contact surface 131 of the foot support plate 130. In some embodiments of the invention, the foot is stabilized at a desired configuration. In some embodiments, above and below are relative to a planned location of that part of the foot during a gait phase.

In some embodiments of the invention, one or more sensors may be used to identify and/or monitor how much weight is carried by different parts of the orthosis and/or the foot using for example, different ROS curves, different height adjustments or during time, to monitor, for example, any change in compression of a material in use. For example, a sensor in a strut or at a point of attachment of the strut may indicate a load carried by that strut. Optionally or additionally, a sensor attached to the bottom of the foot can indicate weight carried by that part of the foot. In some embodiments of the invention, matching sensors may be provided for the other foot. In some embodiments of the invention, the data collected by the sensors is transmitted by wireless (e.g., Bluetooth) or by wire to a remote component, such as a cellular telephone. Local collection of the data using an electronic box mounted on the orthosis is also an option. Optionally, real time processing of the collected data is used for one or more of determining if a desired gait is being provided, determining that weight limits are not passed and/or detecting patient reaction to changes in load bearing. In some embodiments of the invention, real time alerts (e.g., sound and/or visual) are provided to a user or caretaker. Optionally or additionally, the data is later analyzed by a caregiver such as a physiotherapist or a physician. In some embodiments of the invention, gait data is collected using other means, for example, cameras or other gait tracking systems, including such as known in the art. Matching such collected data to an ROS and/or defining changes based on said ROS may be done manually or automatically.

### Fitting and Adjustment

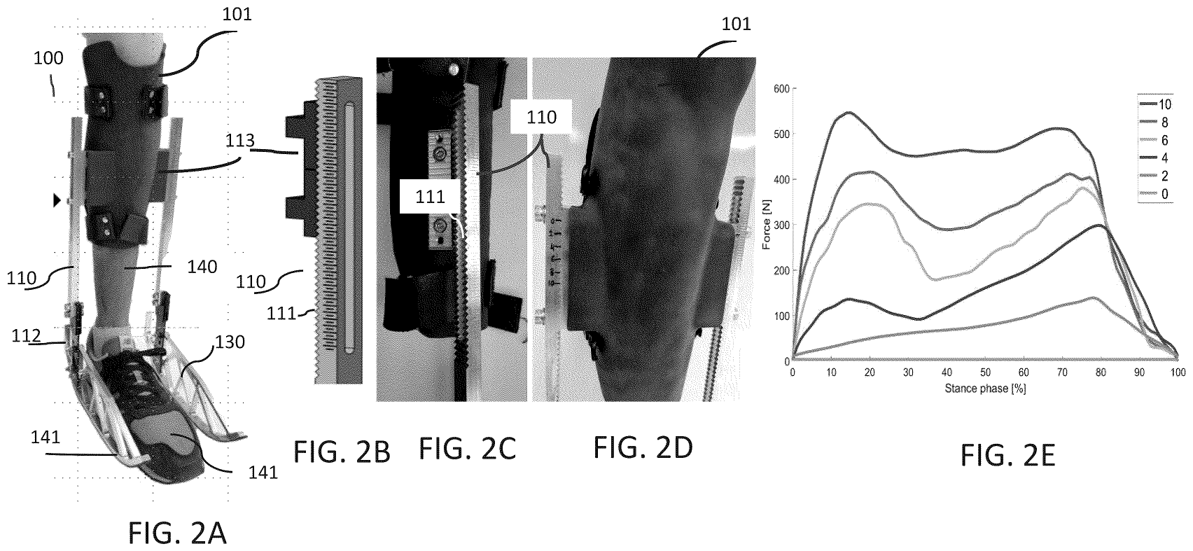

Referring now to FIGS. 2A-E. which show a front image of orthosis 100 mounted on a live foot 141 (FIG. 2A), an enlarged computer generated image (FIG. 1B) of the strut 110 and images of the strut 110 connected to the shank brace 101 via a connector 113 (FIGS. 1C and 1D) in accordance with some embodiments of the invention, and a graph showing values of the ground reaction force (GRF) throughout a stance phase of the gait cycle, for different values of height adjustment of the support plates 113 (FIG. 2E)

FIGS. 2A-2D show an exemplary design for an adjustable-shank orthosis, in accordance with some embodiments of the invention

In a specific example, the length of the part of the strut 110 that connects the foot support plate 130 to the brace 101 determines the height above the ground surface of the surface contact plate 131 relative to the height of the plantar foot surface and can be precisely adjusted along the strut. The level of precision is determined by the length of distance units along the strut on which the length is configured to be adjusted. Many methods for length adjustment and locking at a specified length of the strut are known in the art and can be used to achieve a similar result.

In one example, the height adjustment is achieved using serrated struts that can be fastened to each other in predetermined length steps. An alternative method can be a lead screw which translates turning motion into linear motion, allowing to continuously (without discrete steps) control the length of the strut (which determines the height when the device is in use). In a specific embodiment the resolution is in the order of millimeters. This shank length adjustment potentially allows accurate control over the level of weight transferred to the foot and ankle. In one embodiment the resolution is in steps between 1-2 millimeters. In another embodiment the resolution is in steps between 5-20 millimeters, in another embodiment the resolution is in steps smaller than 1 mm. It is noted that the steps need not be uniform in length and/or the telescoping mechanism used for adjusting the relative positions may allow for continuous adjustment.

In the illustration of an exemplary orthosis presented in FIGS. 2A-D, in the case where the foot is not in contact with the ground, 100% of the load is transmitted to the shank through the struts 110 and the brace 101. When the plantar foot surface contacts the ground surface, the ratio of loading between the foot and the shank can be adjusted anywhere between 0% to 100% and controlled by modifying the position of the shank brace 101 along the strut 110. FIG. 2E presents the results of this height change as demonstrated by plotting the values of the ground reaction force (GRF) throughout the stance phase of the gait cycle, for different values of height adjustment. In the example presented in FIGS. 2A-D, the strut 110 and the bracket 113 comprise serrated regions, 111 and 115 which are configured to fasten to each other in different locations along the strut's serrated region 111. The height adjustment is achieved using the serrated regions 111 and 115 on the strut 110 and bracket 113 that can be fastened to each other in 2 mm steps.

In some embodiments of the invention, the orthosis can also measure and monitor the amount of load transmitted to the foot and/or to the shank and/or to the healthy foot. In some embodiments the orthosis can measure foot and leg movement and gait parameters during movement. This can be achieved by measuring the pressure/force under the foot and/or the pressure/force transmitted via the orthosis to the soft tissues of the shank. The measurement can be done by various means, such as pressure sensors under the foot that directly measure the load on the foot, or strain gauges mounted on the orthosis that measure the force transmitted to the shank (and therefore reduced from the foot). The measurement can be recorded on the device, sent to the cloud, and/or presented to the patient and/or the therapist through a mobile device. As discussed above relating to optional additions to the orthosis, other sensors can be added for collecting and monitoring data related to the patients walking gait or disease.

In some embodiments of the invention, the alignment (e.g., translations and/or rotations) of the device with respect to the leg can be adjusted in any of three axes. For examples, referring to FIGS. 1A-C and FIGS. 2A-D, the medial-lateral position and orientation of the contact plates 130 relative to the leg 141 can be adjusted by modulating the brackets 113 connecting between the brace 101 and the struts 101 and/or the brackets 112 connecting between the struts 101 and the foot supports 130.

Optional ways for adjusting and locking the relative position and/or orientation of the device relative to the foot may use, for example, methods known in the art for adjusting relative positions of two objects. Exemplary methods include fastening the screws through elongated slots that allow different positions and/or rotations.

### ROS Curve

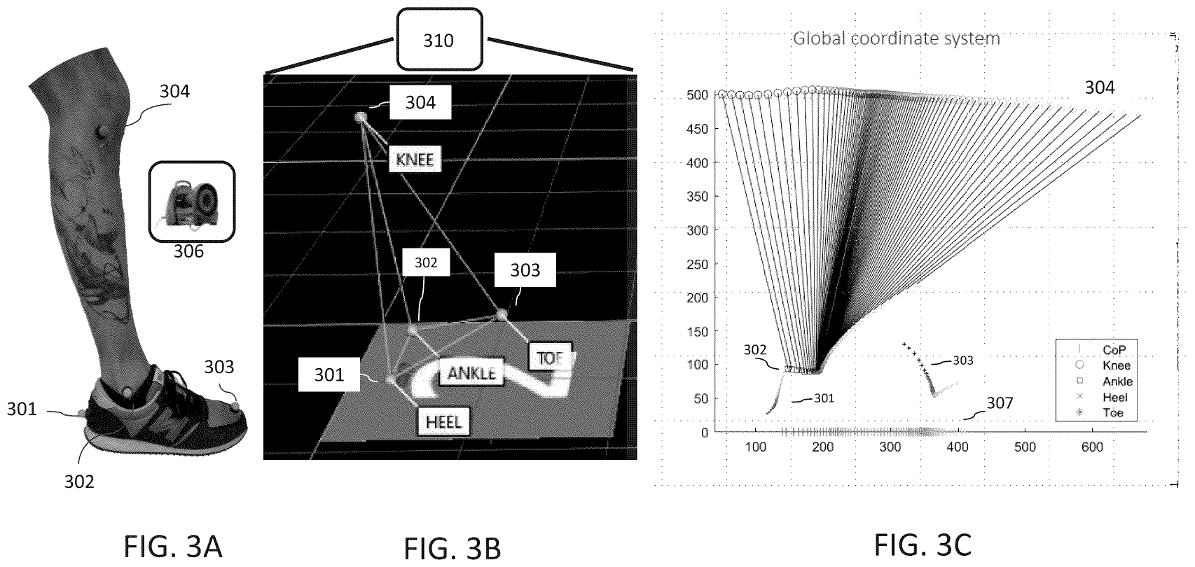

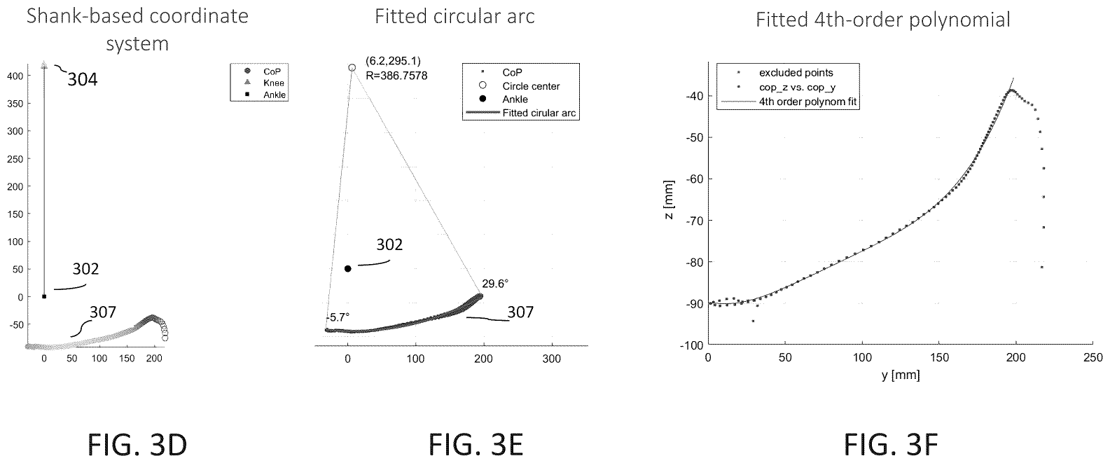

Referring to FIGS. 3A-F which illustrate how to determine and fit a ROS curve to the foot support contact plate 131, by tracking the position of the ankle and knee joints relative to the position of the center of pressure of the foot using a coordinate system during walking (FIG. 3A), using a 3D motion capture system with a floor-embedded force-platform (FIG. 3B) and plotting the captured coordinates in the sagittal plane, relative to the center of pressure (CoP) along time (FIG. 3C).

In an exemplary method, the relative position of the foot center of pressure (CoP) in local coordinate systems affixed to the shank are used to determine and fit the ROS curve. Optionally, the ROS curve is determined using computer aided design (e.g., by running suitable software on a computer, for example, following the methods as described herein. Such software may be provided on a tangible computer-readable media and/or otherwise be stored in memory).

In one exemplary method illustrated in FIGS. 3A-D, the curve is computed by tracking the position of the ankle joint relative to the position of the center of pressure of the foot on the ground during normal walking. As exemplified in FIG. 3A, the 3D positions of reflective markers attached at the heel 301, ankle 302, toe 303 and knee 304 of a subject are measured during walking using a 3D motion capture laboratory 306 equipped with a floor-embedded force-platform (FIG. 3B) (for example, a Vicon motion capture system and/or AMTI force plates can optionally be used). The position of the markers heel 301, ankle 302, toe 303 and knee 304 of FIG. 3A are then captured and illustrated by a software on the computer screen 310 (FIG. 3B). FIG. 3C depicts the positions of the markers of the heel 301, ankle 302, toe 303 and knee 304 of FIG. 3A and the foot center of pressure 307 (CoP) in the sagittal plane in the global coordinate system along time (represented by the passing from blue to yellow). The positions of the markers of the heel 301, ankle 302, toe 303 and knee 304 and CoP 307 are then transformed to the shank-based coordinate system. FIG. 3D presents an example of a single support phase with its origin at the ankle joint as shown in a graph.

The fitting to the ROS curve can be effected in various ways (for an example see, Hansen, Andrew H., Dudley S. Childress, and Erick H. Knox. “Roll-over shapes of human locomotor systems: effects of walking speed.” Clinical biomechanics 19.4 (2004): 407-414, or Bapat G M, Myers S A. A robust technique for optimal fitting of roll-over shapes of human locomotor systems. Medical Engineering & Physics, 100. https://doi(dot)org/10.1016/j.medengphy.2022.103756)

One optional method can be fitting the CoP in the shank-based coordinate system (using markers affixed to knee ankle and CoP) with a circular arc utilizing the center, radius, initial and final angles of the circular arc (FIG. 3E)

In one optional method fitting the CoP in the shank-based coordinate system with a (4th order) polynomial function may be effected (FIG. 3F); or optionally another function or a combination of functions.

In some embodiments, the coordinate system can be assessed using markers affixed to the thigh-shank complex, for example, if a compensation of knee movement is required. The ROS fitting typically results in a curve that is convex throughout (positive curvature with respect to the ankle). In some embodiments, the overall curvature is between 0.1, 0.5 and 0.8 times the person's height. [see, for example, Hansen, Andrew H., Dudley S. Childress, and Erick H. Knox. “Roll-over shapes of human locomotor systems: effects of walking speed.” Clinical biomechanics 19.4 (2004): 407-414; Hansen, Andrew H., and Dudley S. Childress. “Effects of adding weight to the torso on roll-over characteristics of walking.” Journal of Rehabilitation Research & Development 42.3 (2005); Hansen, Andrew H., and Margrit R. Meier. “Roll-over shapes of the ankle-foot and knee-ankle-foot systems of able-bodied children.” Clinical Biomechanics 25.3 (2010): 248-255] . . . .

In some options, the ROS curve can also be used to design a contact plate that does not necessarily roll along a straight line, such that rolling may not be in a plane, but define a three-dimensional curve.

In some options, the ROS curve is adjusted individually, for specific needs, e.g., if other injuries are combined, with the ankle injury e.g. an injured or fixed knee together with an ankle injury.

The ROS curve can alternatively be designed using computer simulations, using neuro-musculo-skeletal models. These models can predict movement patterns based on a mathematical model representing the neuro-musculo-skeletal system (For examples, see De Groote, Friedl, and Antoine Falisse. “Perspective on musculoskeletal modelling and predictive simulations of human movement to assess the neuromechanics of gait.” Proceedings of the Royal Society B 288.1946 (2021): 20202432 or Febrer-Nafría, Míriam, et al. “Predictive multibody dynamic simulation of human neuromusculoskeletal systems: a review.” Multibody System Dynamics 58.3 (2023): 299-339). This is particularly useful because patients who need to be fitted with the device can no longer walk normally, therefore their healthy walking patterns cannot be measured. Using this approach, their optimal ROS can be predicted using simulations instead of experimentally measured.

In some options, the ROS curve can be designed using statistical data. A database including the positions of anatomical landmarks (e.g., ankle and knee) and the positions of CoP throughout the gait cycles can be used to determine their ROS curves. The statistical analysis of the ROS curves for a large number of individuals can then be done to obtain a statistical prescription. For example, the statistical analysis can compute various correlations between anthropometric and physiological characteristics of the individual (e.g., age, sex, height, weight, BMI, foot length, shank length, thigh length, activity level and/or heartbeat) and their ROS. Then, these correlations can be used to fit a ROS to a patient based on their available metrics.

In an exemplary exercise, statistical analysis was performed using previously published open data sets of gait experiments. To identify suitable data sets, data sets were searched for containing the following measurements: position of key points on the leg (e.g., ankle joint, knee joint, hip joint, heel, toe, etc.), position of the CoP, the ground reaction force, the walking speed, and the participants' anthropometric data. The minimum data that allows us to compute an ROS is the ankle and knee positions and the CoP. Four main data sets were identified, consisting of a total of 304 participants:
1. Moreira, Luís, et al. “Lower limb kinematic, kinetic, and EMG data from young healthy humans during walking at controlled speeds.” Scientific data 8.1 (2021): 103.
2. Schreiber, Céline, and Florent Moissenet. “A multimodal dataset of human gait at different walking speeds established on injury-free adult participants.” Scientific data 6.1 (2019): 111.
3. Lencioni, Tiziana, et al. “Human kinematic, kinetic and EMG data during different walking and stair ascending and descending tasks.” Scientific data 6.1 (2019): 309.
4. Van Criekinge, Tamaya, et al. “A full-body motion capture gait dataset of 138 able-bodied adults across the life span and 50 stroke survivors.” Scientific Data 10.1 (2023): 852.
   From these measurements the ROS curve for every step can be computed and the curves can be analyzed with respect to the walking speeds and the participants' anthropometrics to discover how the parameters affect the ROS.

In some embodiments, a plurality of ready-made models of product parts that can be assembled on the device and are customized to specific subsets of patients, in accordance with general pre-determined properties. These parts can be re-used for more than one patient following recovery and enable the economical manufacture and distribution at a relatively low-cost. Optionally, a predesigned foot-side support component may be prescribed to a patient in accordance to predefined characteristics and assembled to the apparatus

### Foot Support Component

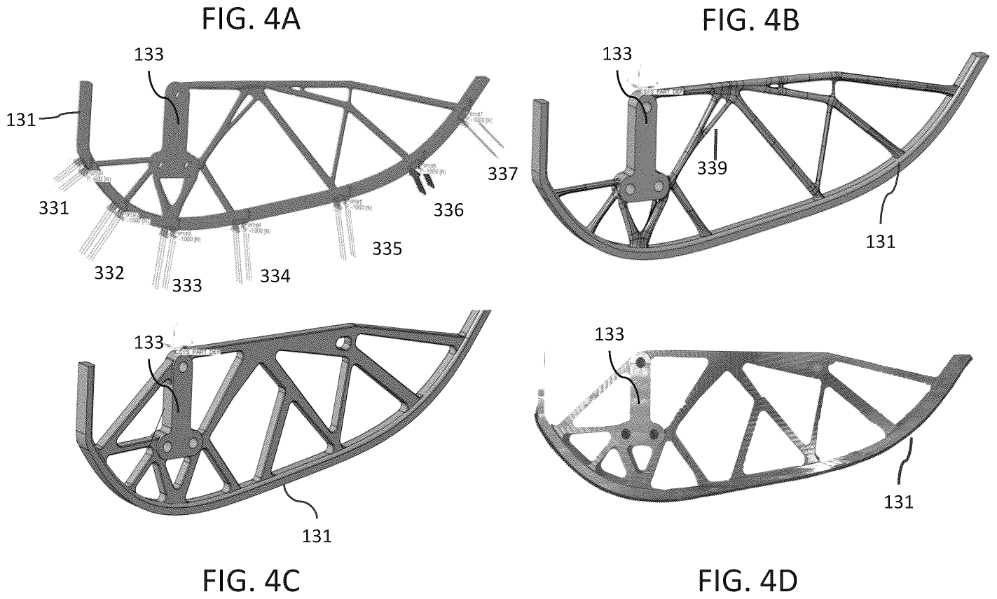

Referring now to FIGS. 4A-D which illustrate stages in a process of designing a foot support 130 in accordance with some embodiments of the invention. Orthosis 100 needs to be moved by the patient and, more specifically, and typically, by a wounded leg. Reducing the weight of such Orthosis can be beneficial for such patients. Standard and semi-standard components may be designed to be light and/or cheap using various engineering methods. A significant portion of the weight (and with a significant moment due to distance from the patient knee & hip) is found in foot support component 130.

In accordance with some embodiments of the invention, the weight of the orthosis device 100 is reduced by reducing the weight of the different components of the orthosis device 100, for example, by changing the shape or structure of the components optionally while employing topology optimization or shape optimization. A computer aided design (CAD) optimization system can be used to define structures and shapes for different materials that would bear the required load during the defined different stages, uses, and conditions.

FIGS. 4A-D depicts an example of such a process that may be effected, for example on the foot support component 130 and contact plate 131. Optionally, a topology optimization procedure performed using for example, the software CREO 9 Generative Design. The optimization can be given multiple load cases, representing the forces transmitted from the ground to the device in different phases during walking. These forces can be provided in various ways, for example, measured during walking, predicted by the neuro-musculo-skeletal simulations described earlier, and/or estimated by statistical analysis of walking gait database. In this example, the objective function of the optimization is to minimize the weight of the device under the constraint that the internal stress does not exceed the material's yield stress, with a certain safety factor, to achieve a structurally stable device

Optionally, some regions are constrained to not be modified by the optimization algorithm. In this example these regions are the ROS curve contacting the ground of the contact plate 131 and the bracket 133 used to connect the part to the rest of the AFO For example, FIG. 4A shows the seven load cases 331-337, representing seven instances along the step. At each instant, the magnitude and the direction of the force applied on the part from the ground may be different, as shown. Optionally, the topology optimization algorithm minimizes the weight of the part while considering each of the load cases 331-337 to ensure that the material will not fail or yield at any point and at any time during the walking cycle. FIG. 4B shows an example of the resultant shape obtained from the topology optimization algorithm. Since the optimization results in beams with a semi-circular cross section (e.g., 339), which are difficult and expensive to manufacture, the design can optionally be simplified making it easier to manufacture by projecting the shape on a two-dimensional plane. The result of this exemplary process is shown in FIG. 4C. Finally, FIG. 4D shows the part designed in FIG. 4C, fabricated by machining an aluminum plate, as an example. FIG. 1C shows a different example, in which the part is fabricated by machining a clear acrylic plate. Optionally, the materials considered by the optimization method may be selected among materials suitable from 3D printing and/or for CNC. Cost may also be a consideration

### Shank Brace

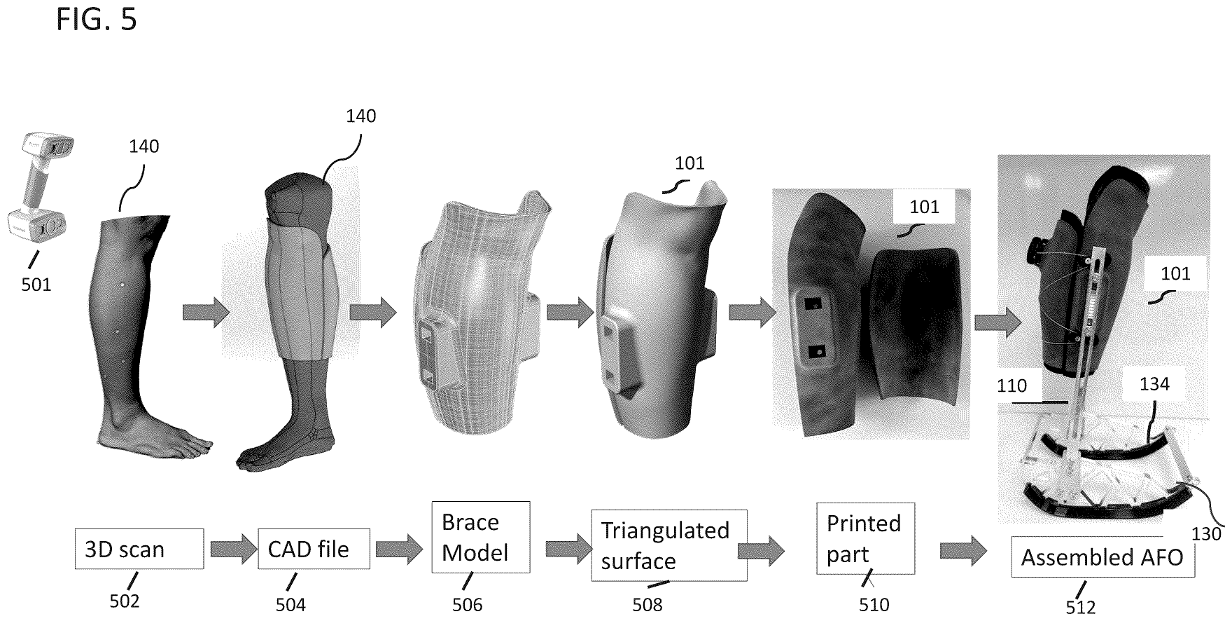

Referring to FIG. 5, which schematically shows a method of fitting an orthosis device 100 to a leg, in accordance with some embodiments of the invention. attaching the orthosis apparatus to the leg can be implemented in various methods that can potentially provide the offloading of weight to be transferred from an area above the ankle. A particular example for designing the shank brace from a 3D scanning data of the leg, and fabrication of a shank brace using selective laser sintering, is provided in FIG. 5.

It is noted that axial stability of attachment can be an important feature, as the axial distance between the bottom of orthosis 100 and the foot bottom plane can affect the load felt by the injured foot, which load may be clinically meaningful. In some embodiments of the invention, a prediction or measurement of axial movement (e.g., at certain loads and/or roll-over curve positions) are used to adjust the actually created rollover curve (possibly by adjusting tread 134 or adding a shim element between cover tread 134 and contact 131, so that the final position of the curve relative to the foot, which is dependent on both the curve and the give/slippage of the brace, are as desired.

Optionally firm attachment is effected through a personalized brace optimized for the specific compromised leg. One option for providing a firm attachment is by designing the brace using a computer aided design 504 (CAD). In this exemplary designing method, the designated desired area of the leg, optionally the shank may be scanned with a scanner 501 and transferred to a computer generated CAD scanned file. Optionally, an algorithm to semi-automatically design the brace from a 3D scan file of the leg can be used to create a model 504 from which a shank brace 101 can be fabricated, optionally by 3D printing. The prepared shank brace can then be assembled 512 with other parts of the orthosis, specifically a strut 110 and a foot plate 130. This method can be beneficial since it can allow accurate, fast, and robust design and fabrication (easily 3D printed. Other optional methods can be manually designing and fabricating the brace through plaster casting of the leg. One potential benefit of 3D scanning is a non-contact technique. An additional potential advantage of scanning is that it allows keeping a digital file of the brace, such that modifications can be made and re-fabricated without requiring to start the entire process from the beginning. Another potential benefit is that the scanning process and the design/fabrication process can be conducted in places that are remote from each other by sending the scanning file directly to the designer.

Referring now to FIGS. 6A-J which are an example of a process for production of an orthosis device in accordance with some embodiments of the invention. FIGS. 6A-J show how the different AFO components and methods used to design them may be integrated and combined into one device. Optionally, the components of the orthosis 100 are fabricated separately and assembled in accordance to the desired use and need of the patient. Exemplary parts that are can be fabricated specifically may be the shank brace 101, the foot plates 130 or the cover treads 134. Optionally, components of the orthosis device may be fabricated according to a predefined design. Pre designed components can be for example, the strut 110 or related brackets 113. As described in the previous sections, the shank brace can optionally be designed using CAD (FIG. 6B) following scanning with a scanning device (FIG. 6A). The design can then be printed or fabricated in accordance to the model prepared by the CAD (FIG. 6C). To design the foot plates the ROS curve is determined. Optionally the ROS is determined per patient using 3D motion capture laboratory equipped with a floor-embedded force-platform (FIG. 6D) and processed using a computer based algorithm (FIG. 6E). In some options the ROS is determined statistically using published data. In some options the ROS curve may be determined using predictions based on computer models. The final design and material of parts of the foot plate, or optionally other parts of the orthosis may then be optionally optimized by using a computer algorithm that considers the forces endured by each element (FIG. 6F) the design is then manufactured (FIG. 6E), and assembled together with the other manufactured parts, including the strut (FIG. 6H) to provide the orthosis device (FIG. 6J). To validate the level of offloading, the load applied directly to the foot can be measured, for example by using instrumented insoles (Pedar, Novel, Germany) that measure the plantar pressure. This measurement is beneficial since it allows the option to examine how the walking load is distributed between the foot and the shank surfaces (FIG. 6I).

Use of the orthosis device of the present invention can be effected in conditions in which full and/or partial offloading of the foot and ankle is required. Particularly, it may be beneficial in cases where gradual adjustment of partial offloading is required in the rehabilitation process, where gradually increasing weight load until it is possible to allow patient to start walking. These cases can occur during a variety of medical conditions and health situations.

Conditions potentially treated using the described devices and/or methods include but are not limited to: plantar pressure ulcers caused by diabetes and other vascularization problems, e.g., Charcot's foot. In these cases partial transfer of weight from the foot, while retaining some of the weight load on the foot may help in slowing disease progression by enabling better blood flow and vascularization. Gradual increase of load bearing on the foot can be adjusted following any improvement in these diseases, e.g. when ulcers or pain is reduced.

For conditions e.g. stress fractures, Clubfoot, rehabilitation from various foot/ankle surgeries, bunion, tendon related injuries, ankle replacements, ankle fusion, fractures or wounds in the foot or ankle, complex regional pain syndrome (CRPS), burns, and other conditions which interfere with foot pressure and/or ankle function, partial offloading of weight from the foot, while retaining some of the weight load on the foot, may be beneficial for reduction of muscle decrease in the injured leg. In these cases change of level of weight offloading can be adjusted for a specific exercise or rehabilitation time frame which can then be adjusted to have less or no weight load on the injured foot for rest period.

It is expected that during the life of a patent maturing from this application many relevant orthosis devices will be developed and the scope of the term orthosis device is intended to include all such new technologies a priori.

As used herein the term “about” refers to plus minus 10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the phrases “substantially devoid of” and/or “essentially devoid of” in the context of a certain substance, refer to a composition that is totally devoid of this substance or includes less than about 5, 1, 0.5 or 0.1 percent of the substance by total weight or volume of the composition. Alternatively, the phrases “substantially devoid of” and/or “essentially devoid of” in the context of a process, a method, a property or a characteristic, refer to a process, a composition, a structure or an article that is totally devoid of a certain process/method step, or a certain property or a certain characteristic, or a process/method wherein the certain process/method step is effected at less than about 5, 1, 0.5 or 0.1 percent compared to a given standard process/method, or property or a characteristic characterized by less than about 5, 1, 0.5 or 0.1 percent of the property or characteristic, compared to a given standard.

The term “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The words “optionally” or “alternatively” are used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the terms “process” and “method” refer to manners, means, techniques and procedures for accomplishing a given task including, but not limited to, those manners, means, techniques and procedures either known to, or readily developed from known manners, means, techniques and procedures by practitioners of the chemical, material, mechanical, computational and digital arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental and/or calculated support in the following examples.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. An orthosis apparatus for offloading weight from a foot of a patient, comprising
a leg brace, sized and shaped to attach to an area above the ankle;
at least one foot support component, comprising a contact plate configured to contact a walking surface; and
a strut, upright connecting the leg brace to the foot support component at a certain length between the brace and the foot support component,
wherein the contact plate is positioned at the side of the foot; and
wherein the orthosis apparatus is configured to offload weight by providing support extending downwards to the contact plate.

2. The orthosis apparatus of claim 1, wherein the contact plate comprises a convex un-flat region, sized and shaped to provide a rolling motion when contacting a surface.

3. The orthosis apparatus of claim 1, wherein the contact plate is further sized and shaped to provide the off loading of weight is rolling motion.

4. The orthosis apparatus of claim 1,
wherein the certain length between the brace and the foot support component is adjustable so as to position the contact plate at a height which can be selected to be above, below or at the foot plantar surface.

5. The orthosis apparatus of claim 1,
wherein the contact plate comprises a convex curve shaped region, sized and shaped to provide a rolling motion when contacting a surface.

6. The orthosis apparatus of claim 1, wherein the convex curved shaped region is shaped and sized to provide a rolling motion in the sagittal plane that reproduces the roll-over shape (ROS) obtained by a non injured leg during walking.

7. The orthosis apparatus of claim 1, wherein the curve shaped region comprises at least two distinct curved regions having a different degree of curvature deviation from an axis.

8. The orthosis apparatus of claim 1, wherein the contact plate additionally comprises a frontal plane oriented-curve configured to reproduce a medial-lateral shift in a center of pressure during walking.

9. The orthosis apparatus of claim 1, wherein the brace is fabricated to fit a patient's leg by use of computer generated design based on scanning the leg.

10. The orthosis apparatus of claim 1, comprising at least 2 foot support components, each configured to be positioned at a different side of the foot.

11. The orthosis apparatus of claim of claim 1, wherein the area above the ankle is located on the shank.

12. The orthosis apparatus of claim 1, wherein the overall curvature of the curve shape is between 0.1, 0.5 and 0.8 times the patients' height.

13. The orthosis apparatus of claim 1, wherein the device further comprises a sensor which collects information indicating the amount of load offloaded from the foot to the leg.

14. A method of offloading weight from a foot of a patient, the method comprising:
suspending the leg of the patient at an area above the ankle in an orthosis apparatus brace, wherein the foot is positioned below the knee;
providing support at the side of the foot with a foot support component having a contact plate, situated at the side of the foot and connected by a strut to the brace and not shadowing said foot from a ground,
wherein the support is provided downright from the brace to the contact plate, thereby offloading weight from the foot to the area above the ankle.

15. The method of claim 14, supporting at least two sides of the foot.

16. The method of claim 14, further comprising adjusting the offloading of weight between the foot and the area above the ankle by positioning the contact plate at a height relative to 1-2 millimeter steps over or under the height of the foot plantar surface.

17. The method of claim 14, further comprising adjusting the offloading of weight between the foot and the area above the ankle by positioning the contact plate at a height relative to about the height of the foot plantar surface, wherein said height effects an offload of between 200 gr and 2 kg off the foot over at least 10% of movement along a roll over curve while said foot takes part in a walking movement.

18. The method of claim of claim 14, wherein the area above the ankle is located on the shank.

19. The method of claim 14, further comprising measuring and/or monitoring the amount of load transmitted to the foot and/or to the shank.

20. The method of claim 14, wherein said contact plate comprises a convex curve shaped region, sized and shaped to provide a rolling motion when contacting a surface.

21. The method of claim 14, further comprising adjusting the offload of weight by one or more of:
positioning the contact plate at a height below the foot plantar surface thereby offloading all of the weight to the area above the ankle;
positioning the contact plate at a height above the foot plantar surface thereby offloading all of the weight to the foot; or
positioning the contact plate at a selected height at about the level of the foot plantar surface, thereby offloading part of the weight from the foot to the area above the ankle;
wherein adjusting the height of the contact plate is effected by changing the length between the brace and the foot support component.

22. The method of claim 14, wherein the selected height at about the level of the foot plantar surface, ranges between 30 millimeters over to 30 millimeters under the plantar foot surface.

23. The method of claim 14, wherein the contact plate is comprises a convex curve shaped region, sized and shaped to provide a rolling motion when contacting a surface.

24. The method of claim 14, wherein the adjusting is provided in steps in accordance to a disease progress, exercise needs and/or rehabilitation level.

25. The method of claim 14, wherein the adjusting of offload step is provided for a defined time frame.

26. An orthosis apparatus for offloading weight from a foot of a patient, comprising:
a leg brace, sized and shaped to attach to an area above the ankle; and
at least one foot support component, comprising a contact plate,
wherein the at least one foot support component is connected to the leg brace;
wherein the contact plate comprises a convex un-flat region, sized and shaped to provide a rolling motion when contacting a surface, and wherein the contact plate is positioned at the side of the foot.
